(19) United States
(12) Patent Application Publication
(10) Pub. No.: US 2025/0391549 A1
(43) Pub. Date: Dec. 25, 2025

(54) Method And System Of Mobile Intelligent Sensor Device To Enumerate And Detect Surgical Tools

(71) Applicant: TaskUnite Inc., Buffalo, NY (US)

(72) Inventor: Bianca Gonzalez, Buffalo, NY (US)

(21) Appl. No.: 19/246,009

(22) Filed: Jun. 23, 2025

Related U.S. Application Data
(60) Provisional application No. 63/662,672, filed on Jun. 21, 2024.

(51) Int. Cl. G16H 40/20; A61B 34/20; A61B 90/00; A61B 90/92; A61B 90/96; G06V 10/44; G06V 10/74; G06V 10/764; G06V 10/94; G06V 20/52

(52) U.S. Cl. CPC G16H 40/20; A61B 34/20; A61B 90/361; A61B 90/92; A61B 90/96; G06V 10/44; G06V 10/74; G06V 10/764; G06V 10/95; G06V 20/52; A61B 2034/2055; A61B 2034/2065; A61B 2090/0805; G06V 2201/034

(57) ABSTRACT

A method that includes capturing, by an imaging and tracking device, an image of a container and performing, by a processor in communication with the imaging and tracking device, image analysis on the image captured by the imaging and tracking device. The method also includes determining, based on the image analysis, one or more of a presence or an absence of inventory items with respect to the container. Responsive to determining the presence of the inventory items, the method includes identifying the inventory items that are contained within the container. Responsive to determining the absence of the inventory items, the method includes identifying the inventory items that are absent from the container.

### CROSS-REFERENCE TO RELATED APPLICATION(S)

This application claims priority to and the benefit of U.S. Provisional Patent Application Ser. No. 63/662,672, filed Jun. 21, 2024, the entire disclosure of which is hereby incorporated by reference.

### TECHNICAL FIELD

This disclosure relates to inventory management, and more particularly, to health care inventory imaging and tracking to monitor, track, and analyze inventory usage in the health care field based on imaging, motion, and/or other detections.

### BACKGROUND

Accurate inventory tracking is a critical component of hospital operations, directly affecting patient safety, surgical efficiency, and compliance with procedural protocols. Manual enumeration of inventory items in the health care field is a widespread practice, often requiring visual confirmation and hand-counting by medical staff. Current approaches to inventory tracking may rely heavily on human oversight and manual processes, which are time-consuming, error-prone, and difficult to scale. Even small errors in inventory counts can delay procedures or pose safety risks, such as retained surgical items.

By way of example, surgical tools may often be manually counted or otherwise tracked for surgical procedures. For example, surgical tools may be inventoried (e.g., counted, tracked, identified, etc.) before and after surgical procedures to ensure that all surgical tools are accounted for to ensure that no surgical tools are misplaced or inadvertently left on or within a patient. However, such manual inventory techniques may be error-prone and time-consuming for the medical staff supporting the surgical procedures, thereby significantly increasing the risk of time delays and/or safety risks for the patient.

Some industries may opt for intelligent inventory systems. However, conventional intelligent inventory systems may be optimized for container-level stock estimation or object removal detection, and may not be configured to perform high-precision, per-item enumeration of items. These limitations may hinder deployment in the medical industry, such as in operating rooms or surgical preparation areas where dynamic, mobile, and highly accurate enumeration is required.

### SUMMARY

In one aspect of the present disclosure, a method is disclosed. The method includes capturing, by an imaging and tracking device, an image of a container, and performing, by a processor in communication with the imaging and tracking device, image analysis on the image captured by the imaging and tracking device. The method also includes determining, based on the image analysis, one or more of a presence or an absence of inventory items with respect to the container. Responsive to determining the presence of the inventory items, the method also includes identifying the inventory items that are contained within the container. Responsive to determining the absence of the inventory items, the method also includes identifying the inventory items that are absent from the container.

In some implementations, the method includes detecting, by the imaging and tracking device a triggering condition. The image is captured by the imaging and tracking device in response to the triggering condition.

In some implementations, the triggering condition includes one or more of motion detection, vibration detection, or weight changes.

In some implementations, responsive to determining the presence of inventory items, the method further includes determining, based on the image analysis, a distance between the inventory items that are contained within the container.

In some implementations, determining one or more of the presence or the absence of the inventory items includes comparing the image captured by the imaging and tracking device to a predefined configuration of the container. The predefined configuration includes one or more of a predefined quantity of the inventory items or a predefined location of the inventory items.

In some implementations, identifying the inventory items that are contained within the container includes identifying the inventory items based on one or more features of the inventory items. The one or more features include at least one of color, shape, size, relative position, a barcode, or a label.

In some implementations, the method includes detecting labels of the inventory items that are contained within the container and classifying the inventory items that are contained within the container based on the labels.

In some implementations, the method includes generating an alert and transmitting the alert to an inventory management system for one or more of storage, display, or order initiation. The alert includes identification of one or more of the inventory items that are contained within the container or the inventory items that are absent from the container.

In some implementations, the method includes determining a field of view of an image sensor of the imaging and tracking device based on the image analysis on the image captured and comparing the field of view to a predefined threshold condition. Responsive to determining that the field of view corresponds to the predefined threshold condition, the method includes articulating the imaging and tracking device via a mount to adjust the field of view. The method also includes capturing, by the imaging and tracking device, an additional image of the container, wherein the image analysis is performed on the additional image.

In some implementations, the image analysis includes performing, by a machine learning model, pixel quantification of the container, and applying, using the processor, object detection based on one or more of physical features of the inventory items or data present on the inventory items.

In some implementations, the data includes one or more of labels or barcodes.

In another aspect of the present disclosure, a system is disclosed. The system includes an imaging and tracking device that is configured for removable mounting to a container via a mount and a server device in communication with the imaging and tracking device. The imaging and tracking device is configured to capture an image of the container. The server includes a processor that is configured to perform an image analysis on the image captured by the imaging and tracking device and determine, based on the image analysis, one or more of a presence or an absence of inventory items with respect to the container. Responsive to determining the presence of the inventory items, the processor is also configured to identify the inventory items that are contained within the container. Responsive to determining the absence of the inventory items, the processor is also configured to identify the inventory items that are absent from the container.

In some implementations, the imaging and tracking device is further configured to detect a triggering condition. The imaging and tracking device is configured to capture the image in response to the triggering condition.

In some implementations, responsive to determining the presence of inventory items, the processor is further configured to determine, based on the image analysis, a distance between the inventory items that are contained within the container.

In some implementations, determining one or more of the presence or the absence of inventory items includes comparing the image captured by the imaging and tracking device to a predefined configuration of the container. The predefined configuration includes one or more of a predefined quantity of the inventory items or a predefined location of the inventory items.

In some implementations, identifying the inventory items that are contained within the container includes identifying the inventory items based on one or more features of the inventory items. The one or more features include at least one of color, shape, size, relative position, a barcode, or a label.

In some implementations, the mount includes an articulating device that is configured to one or more of translate the imaging and tracking device with respect to the container or rotate the imaging and tracking device with respect to the container.

In some implementations, the processor is further configured to determine a field of view of an image sensor of the imaging and tracking device based on the image analysis on the image captured and compare the field of view to a predefined threshold condition. Responsive to determining that the field of view corresponds to the predefined threshold condition, the processor is also configured to articulate the imaging and tracking device via the articulating device to adjust the field of view. The processor is also configured to capture, by the imaging and tracking device, an additional image of the container. The image analysis is performed on the additional image.

In some implementations, the image analysis includes performing, by a machine learning model, pixel quantification of the container, and applying object detection based on one or more of physical features of the inventory items or data present on the inventory items.

In another aspect of the present disclosure, a method is disclosed. The method includes removably coupling an imaging and tracking device to a container via a mount. The mount includes an articulating device that is configured to articulate the imaging and tracking device. The method also includes detecting, by one or more of the imaging and tracking device or the mount, a triggering condition. Responsive to detecting the triggering condition, the method includes capturing, by the imaging and tracking device, an image of the container. The method also includes comparing, by a processor in communication with the imaging and tracking device, the image captured by the imaging and tracking device to a predefined configuration of the container to determine one or more of a presence or an absence of inventory items with respect to the container. The inventory items are surgical tools. The predefined configuration includes one or more of a predefined quantity of the inventory items or a predefined location of the inventory items. Responsive to determining the presence of the inventory items, the method also includes identifying the inventory items that are contained within the container. Responsive to determining the absence of the inventory items, the method also includes identifying the inventory items that are absent from the container.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a block diagram illustrating an example of an inventory imaging and tracking intelligence system.

FIG. 2 is a block diagram illustrating an example of an imaging and tracking device used in an inventory imaging and tracking intelligence system.

FIG. 3 is a block diagram illustrating an example of an imaging and tracking device coupled to a container for monitoring and tracking inventory items.

FIG. 4A illustrates a perspective view of an imaging and tracking device coupled to a container in a first position.

FIG. 4B illustrates a perspective view of the imaging and tracking device of FIG. 4A coupled to the container in a second position.

FIG. 5A illustrates a perspective view of an example of inventory items contained within a container.

FIG. 5B illustrates a perspective view of the example of inventory items shown in FIG. 5A having boundary boxes defined for the inventory items.

FIG. 6 is a block diagram illustrating an example of a workflow of an inventory imaging and tracking intelligence system.

FIG. 7 is a block diagram illustrating an example of a computing device which may be used in an inventory imaging and tracking intelligence system.

FIG. 8 is a flowchart illustrating a first example of a technique for detecting the presence or absence of inventory items using an inventory imaging and tracking intelligence system.

FIG. 9 is a flowchart illustrating a second example of a technique for detecting the presence or absence of inventory items using an inventory imaging and tracking intelligence system.

FIG. 10 is a flowchart illustrating a third example of a technique for detecting the presence or absence of inventory items using an inventory imaging and tracking intelligence system.

### DETAILED DESCRIPTION

Implementations of this disclosure include using an imaging and tracking device for real-time health care inventory intelligence. The imaging and tracking device includes an image sensor and a processor. The image sensor captures images of inventory items, such as medical supplies and/or medical equipment (e.g., surgical tools), stored within a furniture unit (e.g., a container, cart, shelving unit, etc.) to which at least a portion of the imaging and tracking device is coupled. The processor processes the images captured using the image sensor to one or more of detect the presence and/or absence (e.g., absence due to retrieval from the furniture unit and/or lack of inventory) of the inventory items, identify the inventory items, determine a distance between the inventory items present, or generate a signal including data associated with the inventory items present and/or absent. A software application running on a server device uses the signal to automatically update a database record associated with the inventory items within a database. Information associated with the updated database record is then transmitted to a client device in communication with the server device. The information may include instructions for rendering a graphical user interface of the software application at the client device.

An imaging and tracking device as disclosed herein is capable of capturing information indicative of a current inventory quantity within or on a furniture unit in real-time through one or more sensors (e.g., an image sensor, a motion sensor, a pressure sensor, or another sensor, or a combination thereof). For example, the sensors may be used to recognize inventory items stored within or on the furniture unit based on detected appearances of the inventory items (e.g., item shapes, color, text content, graphic content, item sizes, or the like) and/or locations of those inventory items within or on the furniture unit. Information recorded using the sensors is used in real-time to detect the presence and/or absence of the inventory items with respect to the furniture unit. For example, in some implementations, enumerations of the inventory items stored within or on the furniture unit may be used to detect a physical retrieval of an inventory item from the furniture unit. The detection of the physical retrieval of the inventory item is used in an automated updating of a database record by a software application to enable real-time tracking of the inventory item. In another example, enumerations of the inventory items present within or on the furniture unit may be compared to a predefined threshold (e.g., predefined parameters, such as predefined quantities of one or more types of inventory items, predefined locations of one or more of the inventory items, etc.)

The real-time detection of changes to inventory items stored within or on a furniture item represents an improvement in inventory tracking computing technology, for example, based on the sensors included within the imaging and tracking device, the processing capabilities of the imaging and tracking device and/or the server which runs the software application (e.g., in enumerating inventory items using real-time image data and detecting changes to inventory items based on such enumerations), and based on other improvements demonstrated throughout this disclosure. The updating of database records associated with inventory items detected to have changed (e.g., by a physical retrieval or absence thereof from a furniture unit) further represents a solution rooted in the technical environment presented by the imaging and tracking device and communicated server to the technical problem of supporting real-time tracking.

In addition to the above improvements, the imaging and tracking device may be removably coupled to various furniture units. For example, the imaging and tracking device may be removably coupled to furniture units via a mount. The mount may facilitate easy connection and disconnection of the imaging and tracking device with respect to the furniture units. As such, the imaging and tracking device may be fit to any furniture unit to enable tracking of the inventory (e.g., presence and/or absence thereof) using an imaging and tracking intelligence system (hereinafter “system”). The mount may further include a robotic component (e.g., an actuating device), which may articulate (e.g., translate and/or rotate) the imaging and tracking device to better capture images of the furniture unit. As such, the mount and the imaging and tracking device may enable a cost-effective manner to track the inventory count and/or inventory volume of existing furniture units (e.g., via retrofitting) without needing to replace existing furniture units.

In some configurations, the imaging and tracking device may detect a triggering condition, such as motion or a lapse of time-based interval, and in response to the triggering condition, capture an image of the furniture unit. The image may be transmitted to a server device that performs an image analysis on the image (e.g., based upon instructions executed by a processor of the server device). In some configurations, the image analysis may be performed by the imaging and tracking device. For example, the image analysis may be or may include detection and/or classification of inventory items based on identification of one or more features of the inventory items, such as, but not limited to, color, shape, size, spatial proximity to known features of the furniture unit containing the inventory items (e.g., walls, surfaces, or other features that structurally form the furniture unit), a barcode (e.g., a barcode located on the inventory items), and a label (e.g., a label located on the inventory items).

In some configurations, the imaging and tracking device and/or the server device may execute a machine learning model to perform pixel quantification of the captured image to analyze pixel-level distributions with the captured image to identify the inventory items that are present within the furniture unit and/or the inventory items that are absent from the furniture unit. As such, the imaging and tracking device and/or the server device may, in some cases, determine an indication of whether the furniture unit is full, partially full (and, optionally, to what extent), or empty. Data associated with such determinations may be stored in a database (e.g., a database of the server device) or used to initiate actions, such as alerting a user and/or reordering inventory items. Examples of machine learning models that perform pixel quantification can be found in U.S. patent application Ser. No. 19/200,255, filed on May 6, 2025, the entire contents of which are incorporated herein for all purposes.

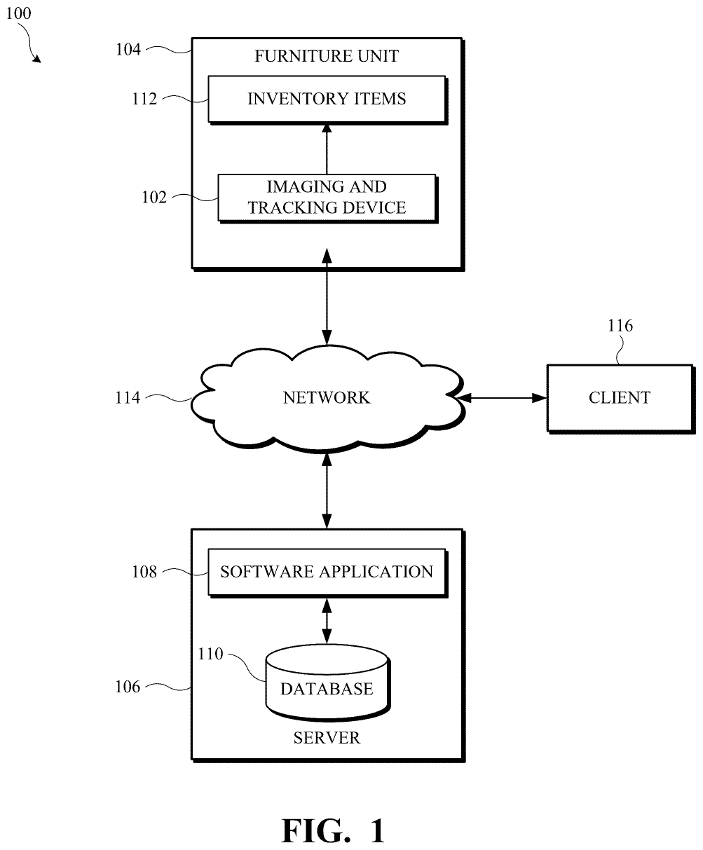

To describe some implementations in greater detail, reference is first made to examples of hardware and software structures used to implement a real-time health care inventory imaging and tracking intelligence system. FIG. 1 is a block diagram showing an example of a real-time health care inventory imaging and tracking intelligence system (e.g., the system 100). The system 100 may include an imaging and tracking device 102 coupled to a furniture unit 104 and a server 106 that runs a software application 108 and stores a database 110.

The imaging and tracking device 102 is a device which is used to monitor inventory items 112 stored within or on the furniture unit 104. The furniture unit 104 is or includes a piece of furniture with at least one surface configured for storing the inventory items 112. In some implementations, the furniture unit 104 may include a number of shelves of the same or different sizes. In some implementations, the furniture unit 104 may include a number of drawers of the same or different sizes. In some implementations, the furniture unit may include a number of cabinets of the same or different sizes. In some implementations, the furniture unit 104 may include a combination of shelves, drawers, and/or cabinets. The furniture unit 104 may be configured to store the inventory items 112 at particular temperatures. For example, the furniture unit 104 may be a refrigerated unit. In another example, the furniture unit 104 may be a heated unit. It will be understood that, aside from the foregoing examples and implementations, the furniture unit 104 may include other types of open or enclosed surfaces or sets of surfaces within or upon which the inventory items 112 may be stored.

The inventory items 112 are items which may be used to provide health care support to a patient. Examples of the inventory items 112 include, but are not limited to, surgical tools, bandages, gauze materials, syringes, medications, ointments, needles, intravenous delivery mechanisms, fluids, medical tapes, and other materials. The inventory items 112 are stored within or on the furniture unit 104. For example, where the furniture unit 104 is a shelving unit with a number of containers, each container of the furniture unit 104 can store some of the inventory items 112. In another example, some of the inventory items 112 may be stored within some of the containers of the furniture unit 104, while other containers of the furniture unit 104 may not store the inventory items 112.

The imaging and tracking device 102 may include an image sensor, a processing component configured to process data captured using the image sensor, a network interface for communicating information processed using the processing component to other devices (e.g., the server 106), and a power source for supplying power for use by the image sensor, the processing component, and the network interface. The imaging and tracking device 102 monitors activity occurring with respect to the furniture unit 104, such as to detect when one of the inventory items 112 is removed from the furniture unit 104, to detect the presence of one or more of the inventory items 112, and to identify the inventory items 112 that are present within and/or absent (e.g., due to removal) from the furniture unit 104. In some implementations, the imaging and tracking device 102 may use sensors other than an image sensor to detect and identify the inventory items 112 present and/or absent. For example, the imaging and tracking device 102 may include a motion sensor. In another example, the imaging and tracking device 102 may include an accelerometer or other sensor capable of detecting vibrations to which the furniture unit 104 is exposed. In yet another example, the imaging and tracking device 102 may include a pressure sensor usable to detect weight changes within the furniture unit 104, which may be associated with the presence and/or absence of the inventory items 112.

The imaging and tracking device 102 may be removably coupled to a portion of the furniture unit 104. For example, the imaging and tracking device 102 may be coupled to a portion of the furniture unit 104 using a mount, whereby the mount may be coupled to the furniture unit 104 via a hook and loop fastener, an adhesive strip, a mounting mechanism which enables the removal of the imaging and tracking device 102 from the furniture unit 104 (e.g., a releasable clamp), or another removable coupling technique. Alternatively, the imaging and tracking device 102 may be permanently coupled to a portion of the furniture unit 104. For example, the imaging and tracking device 102 may be installed using screws or other mechanical fasteners, an adhesive, a mounting mechanism which prevents the removal of the imaging and tracking device 102 from the furniture unit 104 (e.g., a fixed bracket), or another permanent coupling technique.

The server 106 may be a computing aspect that runs the software application 108. The server 106 may be or include a hardware server (e.g., a server device), a software server (e.g., a web server and/or a virtual server), or both. For example, where the server 106 is or includes a hardware server, the server 106 may be a server device located in a rack, such as of a data center.

The software application 108 may be used to process information received from the imaging and tracking device 102, for example, over a network 114. In some implementations, the software application 108 may be used to process information received from the imaging and tracking device 102 to identify the inventory items 112 which are not present on or within the furniture unit 104. In some implementations, the software application 108 can be used to update database records associated with the inventory items 112. In some implementations, the software application 108 can be used to transmit signals indicative of updated database records to a client 116. In some implementations, the software application 108 may be a web application run within a web page served by the server 106 and accessed, for example, by the client 116. In some implementations, the software application 108 may be a mobile application which may include a server-side application running on the server 106 and a client-side application running on the client 116.

The software application 108 may access the database 110 stored on the server 106 to perform at least some of the functionality of the software application 108. The database 110 may be a database or other data store used to store, manage, or otherwise provide data used to deliver functionality of the software application 108. The database 110 may, for example, be a relational database management system, an object database, an XML database, a configuration management database, a management information base, one or more flat files, other suitable non-transient storage mechanisms, or a combination thereof.

The database 110 may store records relating to inventory supplies (e.g., the inventory items 112) which are or may be monitored using the imaging and tracking device 102 or by a different imaging and tracking device within the furniture unit 104 or within a different furniture unit. The database 110 may also store records relating to the usage, including pre-care and post-care instructions, for some or all of the inventory items 112. The database 110 may also store records related to administrative tasks, patient-related tasks, patient names, staff members authorized to retrieve the inventory items 112 from the furniture unit 104, and/or other records.

The software application 108 may include a dashboard which enables a user thereof (e.g., a user of the server 106 or a user of the client 116) to review information processed using the system 100. For example, the dashboard may be used to review information received at the software application 108 from the imaging and tracking device 102. In another example, the dashboard may be used to review changes made to records within the database 110 based on the information received from the imaging and tracking device 102. In yet another example, the dashboard may be used to view information (e.g., knowledgebase articles or the like) associated with the inventory items 112 which may have been detected as being physically retrieved from the furniture unit 104.

The imaging and tracking device 102 may communicate with the server 106 over the network 114. The network 114 may, for example, be a local area network, a wide area network, a machine-to-machine network, a virtual private network, or another public or private network. Communication over the network 114 may use one or more network protocols, such as using Ethernet, TCP, IP, power line communication, Wi-Fi, Bluetooth®, infrared, GPRS, GSM, CDMA, Z-Wave, ZigBee, another protocol, or a combination thereof.

The client 116 may be given access to the software application 108. The client 116 may be or include a hardware client (e.g., a client device), a software client (e.g., a web server and/or a virtual server), or both. For example, the client 116 may be a mobile device, such as a smart phone, tablet, laptop, or the like. In another example, the client 116 may be a desktop computer or another non-mobile computer. The client 116 may run a client-side software application or other software to communicate with the software application 108. For example, the client-side software application may be a mobile application that enables access to some or all functionality and/or data of the software application 108. The client 116 may communicate with the server 106 over the network 114.

Implementations of the system 100 may differ from what is shown and described with respect to FIG. 1. In some implementations, the imaging and tracking device 102 may communicate with the server 106 over the network 114 using an intermediary relay. For example, the intermediary relay may be or include network hardware, such as a router, a switch, a load balancer, another network device, or a combination thereof. The intermediary relay may receive information and/or commands from and/or transmit information and/or commands to the imaging and tracking device 102 using one or more network protocols, such as using Ethernet, TCP, IP, power line communication, Wi-Fi, Bluetooth®, infrared, GPRS, GSM, CDMA, Z-Wave, ZigBee, another protocol, or a combination thereof.

In some implementations, the server 106 and the client 116 may each represent computing devices located within a common area. For example, the server 106 and the client 116 may both be computers located within a health care clinic or hospital. In some implementations, the server 106 and the client 116 may be combined into a single computing device. In some implementations, the software application 108 may transmit push notifications, text messages, or other alerts to the client 116 without the client 116 first accessing the software application 108 (e.g., via a webpage or otherwise). For example, the software application 108 may be configured to automatically transmit signals to certain clients, such as using a whitelist or otherwise.

In some implementations, a health care facility may use multiple imaging and tracking devices, which may be the same as or different from the imaging and tracking device 102. For example, each of the multiple imaging and tracking devices may be coupled to a different furniture unit or to different shelves, drawers, or cabinets of the same furniture unit. The software application 108 may be used to receive and process signals from each of the multiple imaging and tracking devices. For example, the software application 108 may identify individual imaging and tracking devices from which data is received, such as within a GUI generated by the software application 108 based on the retrieval of one of the inventory items 112.

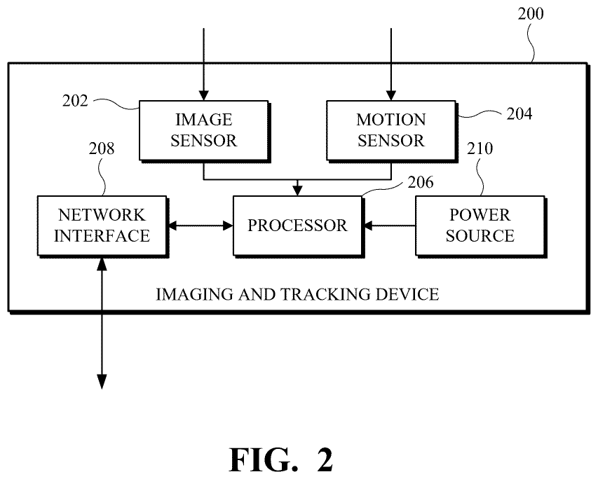

FIG. 2 is a block diagram illustrating an example of an imaging and tracking device 200 used in a real-time health care inventory imaging and tracking intelligence system, for example, the system 100 shown in FIG. 1. For example, the imaging and tracking device 200 may be the imaging and tracking device 102 shown in FIG. 1. The imaging and tracking device 200 may include an image sensor 202, a motion sensor 204, a processor 206, a network interface 208, and a power source 210.

The image sensor 202 may be a sensor configured to capture images within a field of view of the image sensor 202 or otherwise capture data used to construct images. The image sensor 202 may, for example, be a charge-coupled device sensor, an active pixel sensor, a complementary metal-oxide semiconductor sensor, an N-type metal-oxide-semiconductor sensor, or another sensor or combination of sensors.

The motion sensor 204 may be a sensor configured to detect motion within a field of motion of the motion sensor 204. The motion sensor 204 may, for example, be an infrared sensor (e.g., a passive infrared sensor), a microwave sensor, an area reflective sensor, an ultrasonic sensor, or another sensor or combination of sensors.

The processor 206 may be a central processing unit (CPU), such as a microprocessor, and may include single or multiple processors having single or multiple processing cores. In some implementations, the processor 206 may be or otherwise refer to an integrated circuit, for example, a field programmable gate array (e.g., FPGA), programmable logic device (PLD), reconfigurable computer fabric (RCF), system on a chip (SoC), an application specific integrated circuit (ASIC), and/or another type of integrated circuit. The processor 206 may include a cache, or cache memory, for local storage of operating data and/or instructions. For example, the cache may be used to temporarily store data recorded using the image sensor 202, the motion sensor 204, and/or another sensor (e.g., in implementations in which the imaging and tracking device 200 includes such another sensor, such as described below).

The network interface 208 may be used to transmit information and/or commands to and/or receive information and/or commands from one or more devices external to the imaging and tracking device 200. The network interface 208 may provide a connection or link to a network (e.g., the network 114 shown in FIG. 1). The network interface 208 may be a wired network interface or a wireless network interface. The imaging and tracking device 200 may communicate with other devices via the network interface 208 using one or more network protocols, such as using Ethernet, TCP, IP, power line communication, Wi-Fi, Bluetooth, infrared, GPRS, GSM, CDMA, Z-Wave, ZigBee, another protocol, or a combination thereof.

The power source 210 may be a source for providing power to the imaging and tracking device 200. For example, the power source 210 may be an interface to an external power distribution system. In another example, the power source 210 may be a battery, such as a coin-cell battery or another battery. In some implementations, the power source 210 may also provide power to a mount of the imaging and tracking device 200, such as when the mount includes an articulating device that may articulate the imaging and tracking device 200.

Implementations of the imaging and tracking device 200 may differ from what is shown and described with respect to FIG. 2. In some implementations, the motion sensor 204 may be omitted. In some implementations, one or more other sensors may be included. For example, in some such implementations, the imaging and tracking device 200 may include an accelerometer, pressure sensor, or other sensor capable of detecting vibrations. The accelerometer, pressure sensor, or other sensor may be used to monitor vibrations (e.g., indicative of a person accessing a furniture unit to which the imaging and tracking device 200 is coupled, such as by the person opening a door or pulling on a drawer or shelf of the furniture unit).

The implementation of the imaging and tracking device 200 shown in FIG. 2 may include each of the image sensor 202, the motion sensor 204, the processor 206, the network interface 208, and the power source 210 as being included within a single housing or other enclosure. However, in some implementations, the components of the imaging and tracking device 200 may be physically separated into multiple housings or other enclosures, or otherwise separated. For example, in some such implementations, the image sensor 202 and the motion sensor 204 may be included in a first portion of the imaging and tracking device 200 and the processor 206 and the network interface 208 may be included in a second portion of the imaging and tracking device 200. The first portion may be coupled to the furniture unit. The second portion may be external to the furniture unit. In some implementations, the first portion or the second portion may be part of the mount which coupled the imaging and tracking device 200 to the furniture unit.

In some implementations, the power source 210 may cause the network interface 208 to transmit a signal indicating a low power status of the imaging and tracking device 200. For example, a server device running a software application (e.g., the server 106 and the software application 108 shown in FIG. 1) may receive a signal indicating a low power status of the imaging and tracking device 200. The software application may then indicate the low power status, such as to one or more client devices of personnel of the health care provider that uses the imaging and tracking device 200.

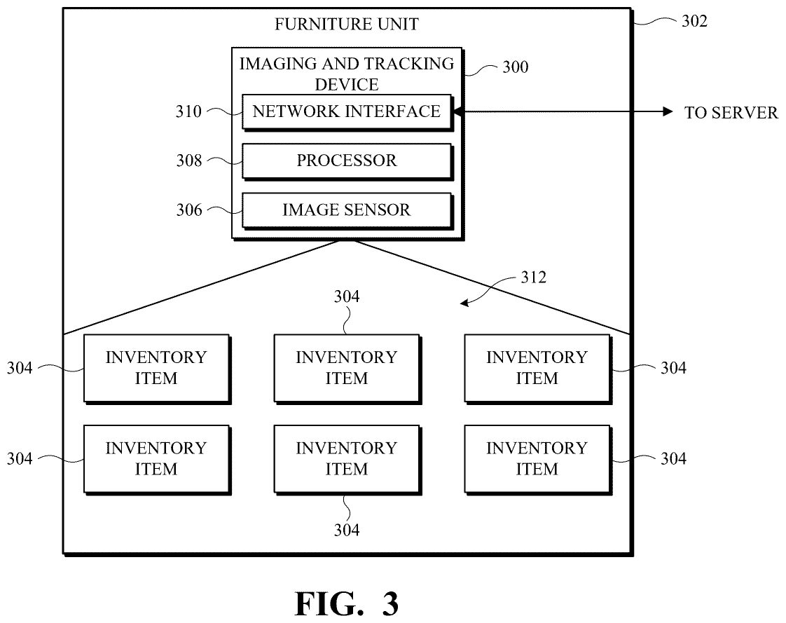

FIG. 3 is a block diagram illustrating an example of an imaging and tracking device 300 coupled to a furniture unit 302 for monitoring and tracking inventory items 304. In particular, an image sensor 306, a processor 308, and a network interface 310 of the imaging and tracking device 300 are shown. The image sensor 306, the processor 308, and the network interface 310 may, for example, respectively be the image sensor 202, the processor 206, and the network interface 208 shown in FIG. 2. In the implementation shown in FIG. 3, the imaging and tracking device 300 may be coupled to the furniture unit 302, and the image sensor 306, the processor 308, and the network interface 310 may be internal to the imaging and tracking device 300 (e.g., within a housing of the imaging and tracking device 300).

The image sensor 306 may have a field of view 312. The field of view 312 may represent the physical area of the furniture unit 302 for which the image sensor 306 may be configured to capture images. In some implementations, the field of view 312 may be adjustable, such as by selectively opening or narrowing aspects of the image sensor and/or by articulating the imaging and tracking device 300 via a mount (e.g., an articulating device of the mount). In some implementations, the image sensor 306 may be included in a controllable mechanism. For example, a user of a software application that processes information received from the imaging and tracking device may use the software application to remotely control, in real-time, a direction of the image sensor 306 via the mount coupling the imaging and tracking device 300 to the furniture unit 302. Changing the direction of the image sensor 306 may thus cause the specific location of the field of view 312 to change.

Implementations of the imaging and tracking device 300 may differ from what is shown and described with respect to FIG. 3. In some implementations, one or more sensors external to the imaging and tracking device 300 may be coupled to the furniture unit 302. For example, the one or more sensors may include weight or pressure sensors configured to detect differences in an amount of weight or pressure applied to a surface on which the inventory items 304 are stored. Such a weight or pressure sensor may be used to detect the physical retrieval of one or more of the inventory items 304. Similarly, such a weight or pressure sensor may be used to detect the presence and/or absence of the inventory items 304. For example, the data recorded using such a weight or pressure sensor can be processed by the processor 308 to detect if one of the inventory items 304 which should be present (e.g., based on a predefined inventory list) is absent. In some such implementations, a single weight or pressure sensor may be configured to measure changes in weight or pressure for the entire surface of the furniture unit 302. In other such implementations, multiple weight or pressure sensors may each be disposed in a different location about the surface of the furniture unit 302 and configured to measure changes in weight or pressure for their specific locations.

In some implementations, a light source may be used to illuminate all or a portion of the furniture unit 302. For example, where the furniture unit 302 is or includes an enclosed piece of furniture, the image sensor 306 may not be exposed to enough light to effectively capture images for detecting the inventory items 304. In some such implementations, the image sensor 306 and the light source may be included in a common housing or other enclosure.

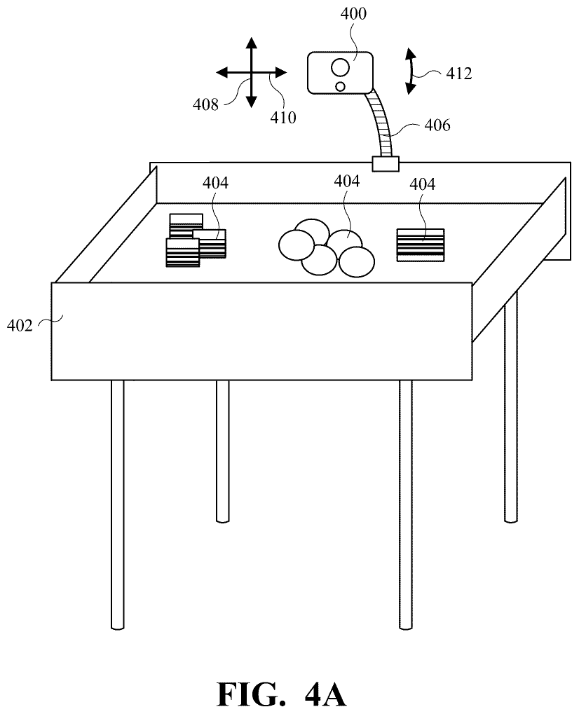

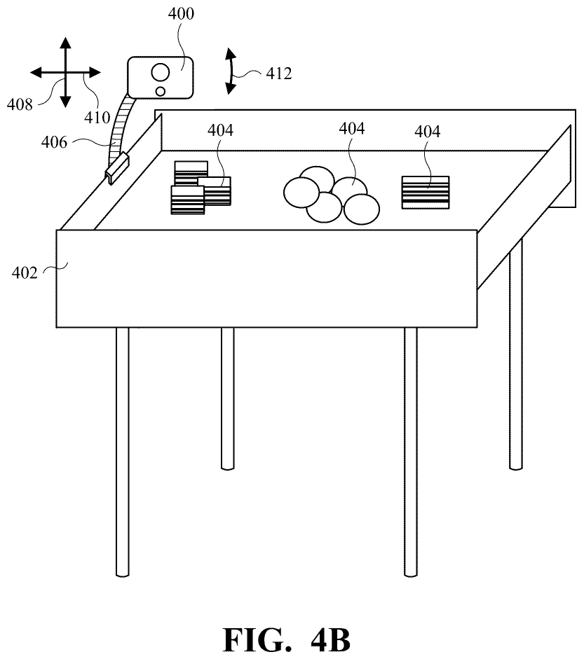

FIG. 4A illustrates a perspective view of an imaging and tracking device 400 coupled to a furniture unit, which in this case may be a container 402 that contains inventory items 404 therein. The imaging and tracking device 400 may be coupled to the container 402 in a first position, such as the position shown in FIG. 4A. Alternatively, or additionally, the imaging and tracking device 400 may be coupled to the container 402 in one or more other positions, such as the second position shown in FIG. 4B.

The imaging and tracking device 400 may be the imaging and tracking device 102 of FIG. 1, the imaging and tracking device 200 of FIG. 2, or the imaging and tracking device 300 of FIG. 3. As such, the imaging and tracking device 400 may be configured to capture images of the container 402 and the inventory items 404 contained therein. As discussed herein, the images of the container 402 captured by the imaging and tracking device 400 may be used to identify the inventory items 404 contained within the container 402 and/or to identify if any of the inventory items 404 are absent from the container 402 in situations where the inventory items 404 should be present within the container 402.

The container 402 may contain any number of the inventory items 404. As shown in FIGS. 4A and 4B, the inventory items 404 may be any size and/or shape. Moreover, the inventory items 404 may contain a variety of features and/or identifiers, which may be used to identify and/or categorize the inventory items 404. For example, the inventory items 404 contained within the container 402 may include various types of items, whereby each type of item may be identifiable by at least one of its size, shape, color, label, barcode, or location (e.g., location within the container 402). As such, the inventory items 404 are not particularly limited to any one item or category of item. By way of example, the inventory items 404 may be, or may include, medical items, such as those described herein, or other hardware items, consumer products, construction materials, sporting goods, food items, beverage items, other items, or a combination thereof. In one example, the container 402 may be a medical cart used in operating rooms. In such a case, the container 402 may be used to organize surgical tools and other medical supplies (e.g., the inventory items 404) needed to perform surgery.

The container 402 may be any type of container that may contain one or more of the inventory items 404. In some implementations, the container 402 may include one or more dividers or sub-containers (e.g., bins, housings, storage units, or the like) within the container 402, whereby the inventory items 404 may be contained in any one of the divided sections of the container 402 and/or any one of the sub-containers. In some configurations, certain types of items may be stored in specific locations within the container 402. For example, in the above example of a medical cart used in operating rooms, it may be critical to locate the inventory items 404 at specific locations within the container 402 to ensure an effective surgical procedure and to ensure patient safety (e.g., by preventing inadvertent use of an incorrect one of the inventory items 404).

The container 402 may traditionally be free of any components that may enable automated inventory imaging and tracking (i.e., imaging and tracking of the inventory items 404 contained in the container 402). That is, the container 402 may be a conventional container that may only be intended to physically support the inventory items 404. Alternatively, in some configurations, the container 402 may include one or more automated mechanical systems, such as conveyors or actuators, which may automate storage and/or retrieval of the inventory items 404. However, in either case, inventory tracking may conventionally be done manually.

To facilitate automated inventory imaging and tracking of the inventory items 404 contained within the container 402, the container 402 may be retrofit with the imaging and tracking device 400. That is, the imaging and tracking device 400 may be removably or fixedly coupled to the container 402. In some implementations, the imaging and tracking device 400 may be removably coupled to the container 402 to facilitate positioning and repositioning of the imaging and tracking device 400 with respect to the container 402. For example, the imaging and tracking device 400 may be coupled initially to the first position shown in FIG. 4A. After reconfiguring (e.g., repositioning) the inventory items 404 within the container 402, the imaging and tracking device 400 may be moved and recoupled to the container 402 at one or more additional positions, such as the second position shown in FIG. 4B, to adjust a field of view of an image sensor of the imaging and tracking device 400 (e.g., the image sensor 306 of FIG. 3). As such, various furniture units, such as the container 402, may be retrofit with the imaging and tracking device 400 to enable automated inventory imaging and tracking. Thus, automated inventory imaging and tracking may be possible in environments that conventionally require manual inventory management, such as an operating room or other similar environments.

A mount 406 may facilitate coupling (e.g., removable or more permanent coupling) of the imaging and tracking device 400 to the container 402. That is, the mount 406 may act as an intermediary component to connect the imaging and tracking device 400 to the container 402. The mount 406 may be coupled to the container 402 in any desired manner, such as using any of the mechanical and/or adhesive connection techniques described herein. By way of example, the mount 406 may utilize a clamp or frictional fit mechanism to engage a lip or edge of the container 402 such that the field of view of the image sensor of the imaging and tracking device 400 may provide a top-down view of the confines of the imaging and tracking device 400, which may include the inventory items 404.

The mount 406 may be or more include one or more robotic components, which may facilitate movement of the imaging and tracking device 400 while coupled to the container 402. For example, the robotic components may be or may include an articulating device, such as a pan-tilt unit, a servo hinge, a rotary actuator, a linear actuator, a ball screw, a telescoping actuator, an articulating joint (e.g., a robotic arm), another type of motor, another type of actuator, or a combination thereof. Thus, the mount 406 may facilitate movement of the imaging and tracking device 400 in any desired direction with respect to the container 402. For example, the mount 406 may facilitate translation (e.g., vertical (i.e., up and down) translation of the imaging and tracking device 400 with respect to the container 402, such as in the direction 408, and/or horizontal (i.e., side to side) translation of the imaging and tracking device 400 with respect to the container 402, such as in the direction 410), rotation (e.g., pan and/or tilt) of the imaging and tracking device 400, such as in the direction 412, or any combination thereof.

In some implementations, the mount 406—and thus the imaging and tracking device 400—may be freestanding. That is, the mount 406 may include or may be connected to one or more legs or bases to support the imaging and tracking device 400 and facilitate operation thereof. In such a case, the mount 406 may be placed adjacent to the container 402 such that the imaging and tracking device 400 may be positioned to monitor the inventory items 404 therein.

The mount 406 may be, may contain, or may be in communication with, additional mechanical components, which may facilitate movement of the imaging and tracking device 400 based upon implementation of the mount 406 (e.g., based upon implementation of the articulating device thereof). For example, the articulating device (e.g., an actuator) of the mount 406 may be in communication with track, guide, or other lateral member that may guide the imaging and tracking device 400 in one or more of the directions shown in FIGS. 4A and 4B based upon actuation by the actuator. As such, the mount 406 may be, or may be part of, a robotic assembly, which may include any number of any number of tracks.

In some implementations, the imaging and tracking device 400 may contain the articulating device (e.g., the actuator) and the mount 406 may guide the imaging and tracking device 400 in any one of the directions shown in FIGS. 4A and 4B based on movement caused by the articulating device. For example, the mount 406 may include, or may be connected to, a track that may guide the articulating device of the imaging and tracking device 400 along the track in the direction 410.

The imaging and tracking device 400 may be configured to monitor at least a portion of the inventory items 404 stored within the container 402. The imaging and tracking device 400 may include the image sensor, a processing component configured to process data captured using the image sensor, a network interface for communicating information processed using the processing component to other devices (e.g., a server), and a power source for supplying power for use by the image sensor, the processing component, and the network interface, such as those described with respect to FIGS. 1-3. In some configurations, the imaging and tracking device 400 may be in communication with an external processing component and/or an external network interface to facilitate transmittal and/or processing (e.g., analyzing) of the images captured by the image sensor. In such a case, the imaging and tracking device 400 may be free of the processing component and/or the network interface.

As discussed above, the imaging and tracking device 400 may be movably coupled to the container 402 via the mount 406. The imaging and tracking device 400 may also be removably coupled to the mount 406. For example, the imaging and tracking device 400 may be coupled to the mount 406 using a hook-and-loop fastener, an adhesive strip, another mounting mechanism which enables the removal of the imaging and tracking device 400 from the mount 406—and thus also removal of the imaging and tracking device 400 from the container 402, or a combination thereof. Alternatively, the imaging and tracking device 400 may be fixed (e.g., permanently coupled) to the mount 406. For example, the imaging and tracking device 400 may be coupled to the mount 406 using screws or other mechanical fasteners (e.g., bolts, clips, etc.), an adhesive, a mounting mechanism which may prevent removal of the imaging and tracking device 400 from the mount 406, or another permanent coupling technique.

The imaging and tracking device 400 may be coupled to the mount 406 in any desired manner to enable movable positioning of the imaging and tracking device 400 to ensure that a field of view of the imaging and tracking device 400 (e.g., the field of view of the image sensor of the imaging and tracking device 400) accurately captures a desired area or region of the container 402 and the inventory items 404 therein. For example, the imaging and tracking device 400 may be positioned to capture images of an entirety of the container 402 and the inventory items 404 therein.

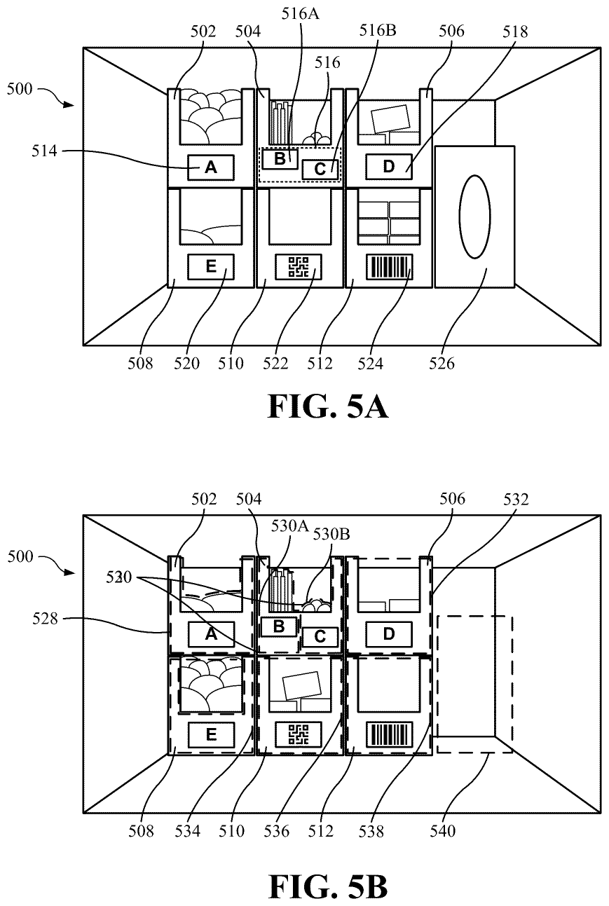

FIGS. 5A and 5B illustrate an example of a containerized environment 500. The containerized environment 500 may be associated with health care inventory management. An imaging and tracking device (e.g., the imaging and tracking device 200 of FIG. 2, the imaging and tracking device 300 of FIG. 3, or the imaging and tracking device 400 of FIGS. 4A and 4B) may be positioned to capture images of the containerized environment 500. The imaging and tracking device may be positioned above or to a side of the containerized environment 500. The imaging and tracking device may capture images of the containerized environment 500 from an overhead (e.g., top-down) view or an angled view. In some implementations, the imaging and tracking device may capture images of multiple containers in a single field of view. The imaging and tracking device may be configured to capture images of the containerized environment 500 in response to detecting a triggering condition.

FIG. 5A shows containers within the containerized environment 500 (e.g., a first container 502, a second container 504, a third container 506, a fourth container 508, a fifth container 510, and a sixth container 512). The containers may be bins that are housed within a furniture unit (e.g., the container 402 of FIGS. 4A and 4B), which may include a cabinet, shelving system, or supply cart used in clinical environments. Each container may include (e.g., hold) one or more inventory items. Each inventory item may include (e.g., exhibit or be associated with) different visual characteristics, for example, volume (e.g., fill level), color, shape, size, spatial proximity to known features of a container containing the inventory item, or arrangement (e.g., a particular way an inventory item is stored in the container, for example, stacked boxes). Containers may include markings covered by inventory items when the fill level is sufficient and more visible when the fill level is insufficient (e.g., low).

A container may include a label (i.e., a first label 514 on the first container 502, a second label 516 on the second container 504, a third label 518 on the third container 506, a fourth label 520 on the fourth container 508, a fifth label 522 on the fifth container 510, and a sixth label 524 on the sixth container 512). The label may be divided into two or more parts, for example, to signify that more than one inventory item is stored in a same container (e.g., the second label 516 is divided into a first label part 516A and a second label part 516B). The label may be readable using optical character recognition (OCR) technology.

For example, the label may include alphanumeric characters (e.g., the first label 514 contains a letter “A,” the first label part 516A of the second label 516 contains a letter “B,” the second label part 516B of the second label 516 contains a letter “C,” the third label 518 contains a letter “D,” and the fourth label 520 contains an “E”). Alternatively, the label may contain a computer-readable (e.g., scannable) image, for example, a Quick Response (QR) code (e.g., the fifth label 522), a barcode (e.g., the sixth label 524), or the like. The label may be facing the imaging and tracking device. The labels may be located within or near the containers and may be captured as part of the image. The labels may be used by the inventory imaging and tracking intelligence system to classify containers and associate containers with specific inventory types.

FIG. 5A represents an image as captured by the imaging and tracking device. The image shown in FIG. 5A may be used as a training image for a machine learning model. Alternatively, the image shown in FIG. 5A may be used as a new image for a machine learning model. Pixel quantification may be performed to determine a status of the containers shown in the image, for example, to determine whether the container is full, partially full, or empty. Similarly, pixel quantification or other object detection techniques, may be implemented to determine whether one or more particular inventory items are present in the containerized environment 500 and/or absent from the containerized environment 500. The image may include one or more containers, their contents (e.g., inventory items), and any visible visual indicators (e.g., labels), without bounding boxes or segmentation overlays.

To further illustrate, the containerized environment 500 may reflect the contents of a mobile cart used in an operating room for surgical procedures. As such, the mobile cart may contain one or more inventory items needed to complete surgery, such as surgical tools and/or medical supplies. In such a scenario, the quantity and/or the placement of each inventory item may be critical to ensure a successful surgery and ensure patient safety. Moreover, the presence and/or absence of any one of the inventory items may also be critical.

Based on the above scenario, the images of the containerized environment 500 may be captured to determine (e.g., calculate and/or estimate) one or more of the following: inventory levels (e.g., quantities of one or more types of inventory items); volumes (e.g., a percentage of any particular container or the mobile cart that is filled by the inventory items; the presence of particular inventory items and/or types of inventory items; the absence of any particular inventory items and/or types of inventory items; the identification of any of the inventory items present within the containerized environment 500 and/or absent from the containerized environment 500; or a combination thereof. Such determinations may be completed by a server device (e.g., the server 106 of FIG. 1) and/or a processor (e.g., the processor 206 of FIG. 2 or the processor 308 of FIG. 3), which may be part of the imaging and tracking device or may be external to the imaging and tracking device (e.g., the processor may be part of the server device).

The system (e.g., the inventory imaging and tracking intelligence system, which may include the imaging and tracking device and/or the server device) may implement various techniques to facilitate the above determinations, such as to determine the absence and/or presence of specific inventory item, such as surgical tools, with the containerized environment 500. These techniques may operate independently or in combination to enhance detection accuracy and robustness under varying environmental and operational conditions.

In some implementations, feature-based object recognition may be utilized. That is, feature-based computer vision algorithms may be used to extract and analyze distinguishing characteristics of the inventory items contained within the captured image, such as those identified above (e.g., shape, size, color, labels, etc.) or other distinguishing characteristics. For example, the system may apply edge detection, segmentation, template matching against a known library of inventory item profiles (e.g., a known library stored on the database 110 of the server 106 shown in FIG. 1), or a combination thereof to analyze the captured image and identify—and thus also differentiate—the inventory items that are present within the containerized environment 500.

In some implementations, the system may utilize machine-readable label detection to identify and/or differentiate the inventory items that are present within the containerized environment 500. For example, the system may implement optical character recognition (OCR) and/or machine-readable code detection algorithms (e.g., barcode or QR code decoding) to extract identifying information directly from labels affixed to or printed on inventory items and/or their associate containers. For example, the fifth label 522 and/or the sixth label 524 may be detected within the captured image to determine that inventory items contained within their respective containers.

In some implementations, the system may be configured to detect unique visual markers (e.g., fiducial markers, colored tags, patterned stickers, etc.) that may be pre-applied to inventory items and/or their respective containers (e.g., the first label 514, the second label 516, the third label 518, or the fourth label 520 may be color-coded or otherwise identifiable by lettering or other fiducial markers). These markers may be used to enable discrete identification of otherwise visually similar objects (e.g., when the containers in the containerized environment 500 are substantially similar in size and/or shape). Such marker detection may utilize a visual tag library (e.g., a visual tag library stored on the database 110 of the server 106 of FIG. 1) and may facilitate identification of inventory items even when under partial occlusion (e.g., blocking).

In some implementations, the system may be configured to identify the absence and/or presence of inventory items based on their spatial relationship to neighboring inventory items and/or based on an expected layout template. For example, the absence of an inventory item may be detected by identifying a vacant region within the containerized environment 500 and/or by identifying a disrupted sequence (e.g., layout) of inventory items within the containerized environment 500. Identifying the vacant region or disrupted sequence may be based on comparing the captured image of the containerized environment 500 to a predefined configuration of the containerized environment 500 (e.g., a predefined configuration of the container). The predefined configuration may be or may include one or more parameters, such as one or more of a predefined quantity of the inventory items or a predefined location of the inventory items. The predefined configuration may be a reference image of the containerized environment 500, which may be compared to the image of the containerized environment 500 captured by the imaging and tracking device.

In some implementations, the system may utilize proximity and depth sensing to detect the presence of inventory items. For example, the imaging and tracking device may include a depth sensor (e.g., a structured light or time-of-flight camera) and/or a short-range proximity sensor. Such sensors may capture depth information (e.g., depth data) of the inventory items contained within the containerized environment 500, which may enable the system to implement three-dimensional segmentation (e.g., partitioning the containerized environment 500 into regions or objects, such as the inventory items or structure of the containers, based on the depth information). Thus, the system may be able to detect stacked or partially occluded inventory items, such as when inventory items (e.g., surgical tools) are moved during use (e.g., during surgery).

In circumstances where the inventory items are manipulated (e.g., during use), the system may employ frame differencing and/or temporal image subtraction techniques to identify changes in inventory item presence over time. For example, the imaging and tracking device may sequentially capture images of the containerized environment 500 over a period of time (e.g., based on a predefined time interval, such as every second, every minute, every 5 minutes, every hour, etc.), whereby a capture image may be compared to a previously captured image (e.g., the image captured immediately before the currently captured image) to identify whether any inventory items have been added and/or removed from the containerized environment 500.

In some implementations, the system may utilize a machine learning model, which may be trained on labeled image datasets of the inventory items. The machine learning model may be capable of learning complex visual patterns to accurately classify various types of inventory items and/or detect the presence of the inventory items even under suboptimal lighting conditions (e.g., a dark environment) and/or partial occlusion of the inventory items.

FIG. 5B illustrates an example of the system identifying the inventory items that are present and/or absent from the containerized environment 500. In particular, FIG. 5B illustrates an image of the containers depicted in FIG. 5A but further includes bounding boxes and segmentation overlays. FIG. 5B may represent an annotated training image, for example, with bounding boxes and annotations provided by a user. The bounding boxes may be drawn by the user or may be generated, for example, using computer vision techniques. The bounding boxes may be verified or adjusted by the user prior to using the image as a training image. The annotations may be stored as metadata within the annotated training image.

Alternatively, FIG. 5B may represent a new image that has been processed by the intelligent vision system to generate bounding boxes. Each container may have one or more bounding boxes (i.e., a first bounding box 528 for the first container 502, a second bounding box 530 for the second container 504, a third bounding box 532 for the third container 506, a fourth bounding box 534 for the fourth container 508, a fifth bounding box 536 for the fifth container 510, and a sixth bounding box 538 for the sixth container 512). The bounding boxes may surround the containers within the image. Each inventory item may be associated with a bounding box, such that containers with multiple inventory items may have multiple bounding boxes (e.g., the second container 504 may have a first bounding box part 530A and a second bounding box part 530B). The bounding boxes may define the boundaries of each container. The bounding boxes may include visual classification of internal regions based on pixel categorization. The bounding boxes may be used to support confidence scoring or further inference about restocking needs.

The bounding boxes may surround (e.g., include or encompass) a container. In some implementations, a bounding box may be segmented to exclude an area corresponding to, for example, a background, the furniture unit (e.g., the mobile cart), or the like. For example, the first bounding box 528 has been segmented to include the first container 502 and an associated inventory item, but to exclude a ceiling and back wall of the furniture unit. Accordingly, for pixel quantification of the first bounding box 528, only pixels corresponding to the first container 502 and the associated inventory item may be considered. In other implementations, a bounding box may be segmented to divide an area corresponding to the container and an area corresponding to the associated inventory item. For example, the fourth bounding box 534 has been segmented to include a first area corresponding to the fourth container 508 and a second area corresponding to the associated inventory item. Accordingly, for pixel quantification of the fourth bounding box 534, a number of pixels in the first area corresponding to the fourth container 508 and a number of pixels in the second area corresponding to the associated inventory item may be used to estimate a volume of the associated inventory item.

FIG. 5B also shows a seventh bounding box 540 corresponding to a location of the inventory item 526. The inventory item 526 is not present in FIG. 5B. Accordingly, the machine learning model may classify substantially all pixels within the seventh bounding box 540 as pixels corresponding to the furniture unit. The seventh bounding box 540 may be generated based on object edges, spatial clustering, or prior training data.

While the example shown in FIG. 5B describes bounding boxes for defining regions around containers, the system is not limited to such regions. In some implementations, the system may identify contours or segmentation regions to enclose inventory items with cylindrical or round shapes, enhancing precision over rectangular bounding boxes. Similarly, for containers with irregular or non-rectangular shapes, the system may define customized segmentation regions based on visual features or training data to enable accurate pixel quantification.

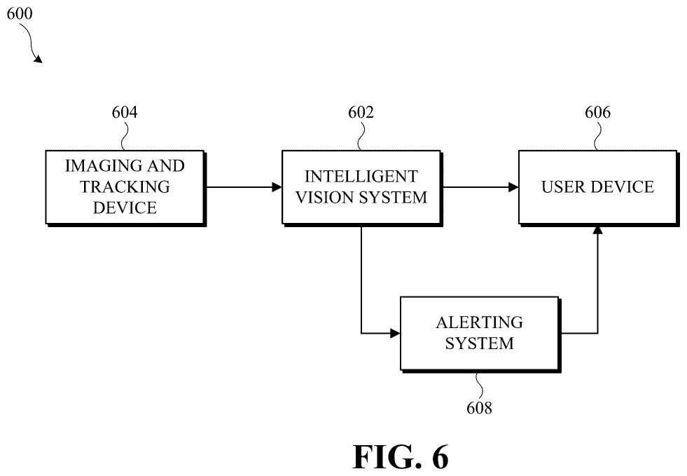

FIG. 6 is a block diagram showing an example of a first workflow 600 of an inventory imaging and tracking intelligence system, hereinafter the system 602. The system 602 may include a processor configured to perform image analysis on image data captured by one or more image devices. For example, the system 602 may be the system implemented to identify the presence and/or absence of inventory items in the containerized environment 500 of FIGS. 5A and 5B utilizing an imaging and tracking device, such as those described with respect to FIGS. 1-4.

The image analysis may involve using computer vision techniques to detect, classify, count, estimate the volume of, or a combination thereof of inventory items contained within a furniture unit, such as those described herein. The image analysis may identify and/or locate inventory items within an image frame of the captured image. Such identified and/or located inventory items may then be counted (e.g., using bounding boxes around detected items, which may include classification labels). The image analysis may further identify and/or locate various barcodes, Quick Response (QR) codes, labels, or other features of specific inventory items in order to identify and/or locate such items. For example, the image analysis may involve implementing any one of the techniques described above with respect to the system of FIGS. 5A and 5B.

By way of example, the system 602 may perform pixel quantification of at least one furniture unit based on image data. The system 602 may be configured to perform pixel quantification using a machine learning model. The machine learning model may be trained on a set of training images labeled with identifiers for one or more predefined inventory items that are contained on or within the furniture unit. For example, in the scenario where the furniture unit is a medical cart that contains one or more surgical tools, the training images may be labeled with particular surgical tools that are located in predefined locations on the medical cart. As such, the system 602 may determine when the predefined inventory items (e.g., the surgical tools) are present and/or absent from the furniture unit (e.g., the medical cart).

Pixel quantification, as used herein, refers to analyzing a furniture unit image by segmenting and classifying pixels into categories, such as inventory item pixels, furniture unit pixels, or, in some implementations, label pixels (e.g., for labels affixed to the furniture unit and/or the inventory items), and calculating the proportion of inventory item pixels to furniture unit pixels within a defined region, such as a bounding box, to determine the present and/or absence of inventory items.

In some implementations, pixel quantification may be used to estimate inventory volume in the furniture unit, for example, by calculating a proportion of the number of inventory item pixels relative to the number of furniture unit pixels. For example, an image of an empty furniture unit may include approximately 100% of pixels corresponding to the furniture unit and approximately 0% of pixels corresponding to the inventory item. In another example, an image of a partially full furniture unit may include approximately 80% of pixels corresponding to the furniture unit and approximately 20% of pixels corresponding to the inventory item. In another example, an image of a full furniture unit may include approximately 50% of pixels corresponding to the furniture unit and approximately 50% of pixels corresponding to the inventory item. Pixel quantification may use a proportion (e.g., ratio or percentage) of pixels corresponding to different objects in the image such that images captured at different distances from the furniture unit may be used to accurately estimate inventory volume.

The system 602 may receive images from an imaging and tracking device 604 (e.g., any one of the imaging and tracking devices described above with respect to FIGS. 1-5B). The imaging and tracking device 604 may be configured to detect a triggering condition. In some implementations, a mount (e.g., the mount 406 of FIGS. 4A and 4B) may be configured to detect the triggering condition. The triggering condition may include motion detection, vibration detection, infrared motion detection, or expiration of a predefined time interval. The imaging and tracking device 604 may be configured to ignore the triggering condition based on specific instructions. For example, the imaging and tracking device 604 may be configured to ignore the triggering condition due to manual override (e.g., such as a technician is restocking shelves), instructions to ignore triggering conditions within a predefined time period after a triggering condition (e.g., after one trigger (e.g., motion), the system enters a “quiet period” where it ignores further triggers for a set time (e.g., 3 minutes)), or the like. The imaging and tracking device 604 may be configured to receive (e.g., from the system 602) an updated triggering condition (e.g., an updated threshold or updated time interval). The imaging and tracking device 604 may replace the triggering condition with the updated triggering condition, and may transmit (e.g., to the system 602) confirmation of the updated triggering condition.

The imaging and tracking device 604 may be configured to capture an image of at least one furniture unit. Capturing an image of the at least one furniture unit may be performed in response to detecting the triggering condition. The imaging and tracking device 604 may include one or more sensors, such as an image sensor (e.g., the image sensor 306), a motion sensor, an infrared sensor, a vibration sensor, or the like. The imaging and tracking device 604 may be removably or permanently coupled to a furniture unit, as described herein. For example, the imaging and tracking device 604 may be retrofit to a furniture unit, such as a medical cart, using the techniques described herein.

The captured image may be transmitted from the imaging and tracking device 604 to the system 602. Transmitting the captured image from the imaging and tracking device 604 to the system 602 may include using a network interface of the imaging and tracking device 604 to wirelessly communicate the captured image to the system 602.

The system 602 may communicate with a user device 606. The user device 606 may be configured to receive the status of the furniture unit from the system 602. That is, the user device 606 may be configured to receive information related to the presence and/or absence of inventory items with respect to the furniture unit and/or information associated with the inventory items (e.g., use instructions, identifying information, quantities, volumes, etc.) The user device 606 may display the status to a user. The user device 606 may be a mobile device or a desktop device. The user device 606 may run a client-side application for viewing inventory data.

The system 602 may be communicatively connected to an alerting system 608. The alerting system 608 may be configured to generate an alert based on the status of the furniture unit. The alert may be generated when the status of the furniture unit meets or falls below a predefined threshold condition. For example, the alert may be generated when one or more of the inventory items are absence from the furniture unit and/or when a quantity of one or more of the inventory items changes from full to partially full, when the quantity of one or more of the inventory items becomes empty, or when the quantity falls below a specified percentage (e.g., 50%, 20%, 0%, or the like). The alerting system 608 may transmit the alert to the user device 606. The alert may notify a user associated with the user device 606 that the furniture unit requires replenishment or that an inventory condition has been met. The alert may be delivered as a push notification, text message, or interface prompt within the client application.

Alternatively, the alert may trigger a signal to an inventory management system to automatically place an order for the inventory item associated with the furniture unit (e.g., when an inventory item is missing). The signal may include information such as an item identifier, a quantity to be ordered, a location of the container, and an urgency level. The inventory management system may process the signal to generate a purchase order, initiate fulfillment with a supplier, or schedule internal restocking procedures. The inventory management system may apply predefined ordering rules based on consumption patterns, threshold levels, or supply chain constraints. In some implementations, user approval may be required to confirm the order before execution. Alternatively, the order may be placed automatically without manual intervention. Automatic inventory ordering may reduce manual inventory tracking and help maintain optimal stock levels. By way of example, in a scenario where the furniture unit is a mobile cart used for surgical procedures, absence of a particular surgical tool (i.e., an inventory item) may be critical to the procedure. In such a case, the alert may automatically trigger personnel to immediately retrieve the necessary surgical tool and bring it to the mobile cart.

Authorized users, such inventory managers, may access the administrative user interface via a client device (e.g., the client 116) to remotely monitor inventory. The user interface may display the annotated image, enabling users to visually assess the status (e.g., inventory status, which may include inventory items present and/or absent) of multiple furniture units simultaneously. The annotated labels facilitate rapid identification of inventory conditions, supporting timely restocking decisions or compliance checks. The system may store the annotated image in the database (e.g., database 110) for record-keeping or transmit it to the user device as part of an alert or status update, enhancing inventory visibility and operational efficiency.

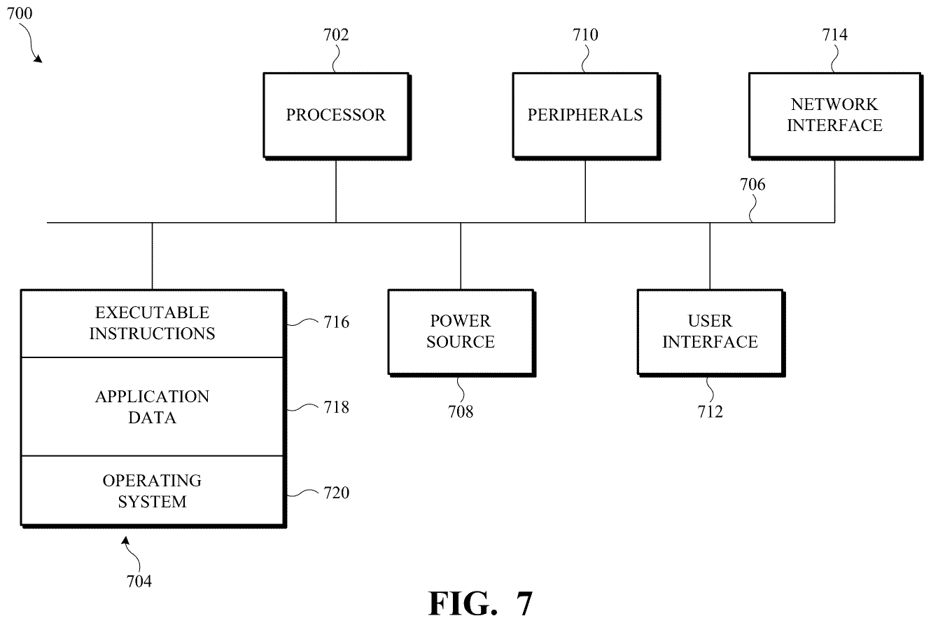

FIG. 7 is a block diagram of an example of a computing device 700 which may be used in an inventory imaging and tracking intelligence system, for example, the system 100 of FIG. 1. The computing device 700 may be used to implement a server on which a software application is run (e.g., the server 106 and the software application 108 shown in FIG. 1). Alternatively, the computing device 700 may be used to implement a client that accesses the software application (e.g., the client 116 shown in FIG. 1). As a further alternative, the computing device 700 may be used as or to implement another client, server, or other device according to the implementations disclosed herein. The computing device 700 includes components or units, such as a processor 702, a memory 704, a bus 706, a power source 708, peripherals 710, a user interface 712, and a network interface 714. One or more of the memory 704, the power source 708, the peripherals 710, the user interface 712, or the network interface 714 may communicate with the processor 702 via the bus 706.

The processor 702 may be a central processing unit (CPU), such as a microprocessor, and can include single or multiple processors having single or multiple processing cores. Alternatively, the processor 702 can include another type of device, or multiple devices, now existing or hereafter developed, configured for manipulating or processing information. For example, the processor 702 can include multiple processors interconnected in any manner, including hardwired or networked, including wirelessly networked. For example, the operations of the processor 702 can be distributed across multiple devices or units that can be coupled directly or across a local area or other suitable type of network. The processor 702 can include a cache, or cache memory, for local storage of operating data and/or instructions.

The memory 704 may include one or more memory components, which may each be volatile memory or non-volatile memory. For example, the volatile memory of the memory 704 can be random access memory (RAM) (e.g., a DRAM module, such as DDR SDRAM) or another form of volatile memory. In another example, the non-volatile memory of the memory 704 can be a disk drive, a solid state drive, flash memory, phase-change memory, or another form of non-volatile (or non-transitory) memory configured for persistent electronic information storage. The memory 704 may also include other types of devices, now existing or hereafter developed, configured for storing data or instructions for processing by the processor 702.

The memory 704 can include data for immediate access by the processor 702. For example, the memory 704 can include executable instructions 716, application data 718, and an operating system 720. The executable instructions 716 can include one or more application programs, which can be loaded or copied, in whole or in part, from non-volatile memory to volatile memory to be executed by the processor 702. For example, the executable instructions 716 can include instructions for performing some or all of the techniques of this disclosure. The application data 718 can include user data, database data (e.g., database catalogs or dictionaries), or the like. The operating system 720 can be, for example, Microsoft Windows®, Mac OS X®, or Linux®; an operating system for a small device, such as a smartphone or tablet device; or an operating system for a large device, such as a mainframe computer.

The power source 708 may include a source for providing power to the computing device 700. For example, the power source 708 can be an interface to an external power distribution system. In another example, the power source 708 can be a battery, such as where the computing device 700 is a mobile device or is otherwise configured to operate independently of an external power distribution system.

The peripherals 710 may include one or more sensors, detectors, or other devices configured for monitoring the computing device 700 or the environment around the computing device 700. For example, the peripherals 710 can include a geolocation component, such as a global positioning system location unit. In another example, the peripherals can include a temperature sensor for measuring temperatures of components of the computing device 700, such as the processor 702.

The user interface 712 includes one or more input interfaces and/or output interfaces. An input interface may, for example, be a positional input device, such as a mouse, touchpad, touchscreen, or the like; a keyboard; or another suitable human or machine interface device. An output interface may, for example, be a display, such as a liquid crystal display, a cathode-ray tube, a light emitting diode display, or other suitable display.

The network interface 714 provides a connection or link to a network (e.g., the network 114 shown in FIG. 1). The network interface 714 can be a wired network interface or a wireless network interface. The computing device 700 can communicate with other devices via the network interface 714 using one or more network protocols, such as using Ethernet, TCP, IP, power line communication, Wi-Fi, Bluetooth, infrared, GPRS, GSM, CDMA, Z-Wave, ZigBee, another protocol, or a combination thereof.

Implementations of the computing device 700 may differ from what is shown and described with respect to FIG. 7. In some implementations, the computing device 700 can omit the peripherals 710. In some implementations, the memory 704 can be distributed across multiple devices. For example, the memory 704 can include network-based memory or memory in multiple clients or servers performing the operations of those multiple devices. In some implementations, the application data 718 can include functional programs, such as a web browser, a web server, a database server, another program, or a combination thereof.

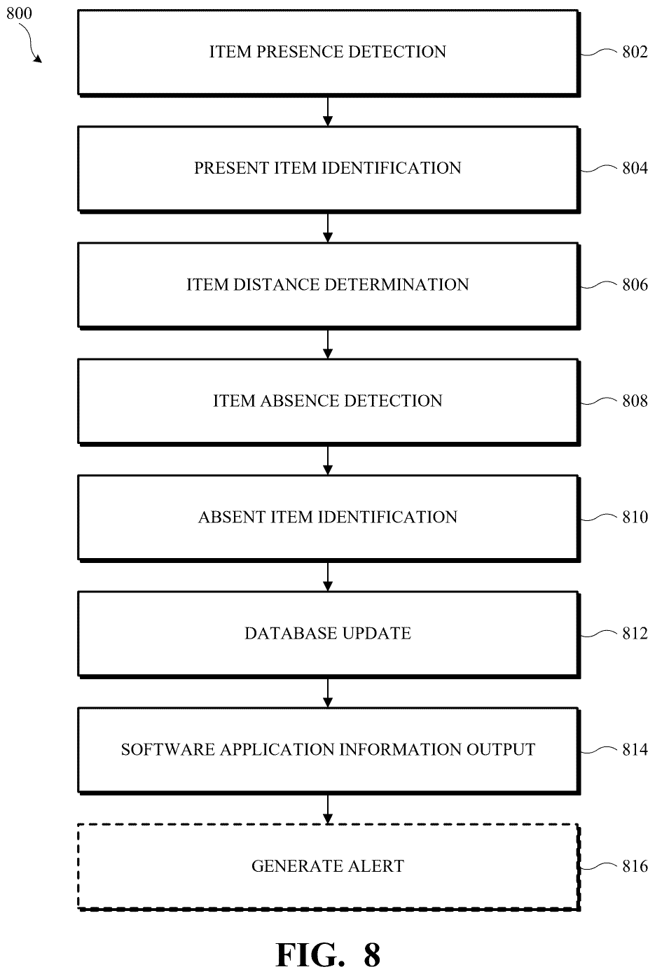

FIG. 8 a flowchart showing an example of a first technique 800 for detecting the presence and/or absence of inventory items using an inventory imaging and tracking intelligence system. The first technique 800 can be executed using computing devices, such as the systems, hardware, and software described with respect to FIGS. 1-7. The first technique 800 can be performed, for example, by executing a machine-readable program or other computer-executable instructions, such as routines, instructions, programs, or other code. The steps, or operations, of the first technique 800 or another technique, method, process, or algorithm described in connection with the implementations disclosed herein can be implemented directly in hardware, firmware, software executed by hardware, circuitry, or a combination thereof.

The first technique 800 may commence with a health care provider creating a task for performance within a software application of a real-time health care inventory imaging and tracking intelligence system (e.g., the software application 108 of the system 100 shown in FIG. 1). Based on the created task, the health care provider may seek to evaluate a furniture unit, such as a mobile cart used in surgical procedures, to determine whether the necessary inventory items, such as surgical tools and/or medical supplies, are present on or within the furniture unit, thereby enabling a successful operation.

At 802, the first technique 800 may detect (e.g., determine) whether one or more inventory items are present on or within the furniture unit. For example, the system may use an imaging and tracking device, such as those described above with respect to FIGS. 1-7, which may capture an image of the furniture unit and the inventory items on or within the furniture unit. The system may then perform image analysis, using one or more of the techniques described herein, to detect the presence of the inventory items.

At 804, the present inventory items may be identified. Identifying the inventory items may include identifying or otherwise determining information usable to identify the inventory items present within the furniture unit, such as from amongst the other inventory items stored on or within the same furniture unit or otherwise used at the same health care facility. The inventory items can be identified by the imaging and tracking device used to detect the presence of the inventory items on or within the respective furniture unit and/or by the software application that receives a signal from the imaging and tracking device. For example, the system may implement one or more of the techniques described above with respect to FIGS. 1-7 to identify the inventory items present on or within the furniture unit, such as by utilized features and/or characteristics of the inventory items (e.g., size, color, shape, location, label presence, barcode presence, etc.).

At 806, the first technique 800 may determine a distance between one or more of the inventory items. For example, the system may implement one or more computer vision techniques to estimate the relative spatial distance between the inventory items positioned within the furniture unit. Such distances may be used to confirm a spatial layout of the inventory items on or within the furniture unit, detect anomalies (e.g., detect item occlusion), implement pattern-based enumeration, optimize storage configurations on or within the furniture unit, or a combination thereof.

By way of example, the distance between inventory items may be based upon pixel quantification or a similar technique. For example, the imaging and tracking device may capture top-down images of the inventory items contained on or within the furniture unit. As such, the system may determine (e.g., calculate or estimate) the distance between inventory items by calculating a distance (e.g., a Euclidean distance) between centroid or boundary keypoints of the inventory items in pixel space. For example, each of the inventory items identified at 804 may be assigned a bounding box or centroid coordinates. The distance between such bounding boxes or centroid coordinates may then be determined.

In some implementations, to determine the distance between inventory items, pixel-based measurements may be mapped. This may be done to map such pixel-based measurements to real-world units (e.g., centimeters). For example, the system may spatially calibrate the imaging and tracking device using known geometric markers and/or fiducial reference points placed on or within the furniture unit. As such, the system may map pixel coordinates to planar world coordinates, enabling real-world distance estimations between the inventory items.

In some implementations, machine learning-based spatial inference may be implemented by the system to determine the distance between inventory items. For example, may utilize a machine learning model (e.g., a machine learning model of the system 602 of FIG. 6) to infer spatial relationships between the inventory items based on contextual visual cues. For example, neural networks trained on labeled datasets (e.g., labeled datasets corresponding to the inventory items and/or one or more features or structures of the furniture unit) may be configured to determine relative distances between the inventory items based on the labeled datasets.

By way of example, consider a medical cart used in an operating room to stage surgical tools for a procedure. An expected layout of the surgical tools may follow a hospital-defined standard such that the surgical tools are arranged with uniform spacing and/or ordered by category (e.g., item type), without overlap. In such a case, the imaging and tracking device may capture images of the medical cart and the surgical tools. The system may then implement a machine-learning model to identify bounding boxes of each surgical tool and compare the bounding boxes to a representation of an expected layout (e.g., a predefined threshold, such as a predefined image of an example of the expected layout). By using such representations of expected layout, the machine-learning model may be trained to effectively identify where the items are located and a spatial proximity of the inventory items to one another.

At 808, the first technique 800 may also detect (e.g., determine) whether one or more inventory items are absent from the furniture unit. For example, the image captured by the imaging and tracking device may be further analyzed (e.g., via a processor of the system) to detect the absence of the inventory items using one or more of the techniques described herein.

At 810, the absent inventory items may be identified. Identifying the absent inventory items may include identifying or otherwise determining information usable to identify the inventory items absent from the furniture unit, such as based on information related to the other inventory items present on or within the furniture unit. The absent inventory items can be identified by the imaging and tracking device used to detect the presence of the inventory items on or within the respective furniture unit and/or by the software application that receives a signal from the imaging and tracking device. For example, the system may analyze the capture image and compare the captured image to a predefined inventory list. That is, the system may compare (e.g., cross-reference) the inventory items present to the predefined inventory list to thus determine which of the inventory items are absent from the furniture unit. In some implementations, the system may identify locations of the furniture unit that are absent inventory items using the techniques described herein. As such, the empty location may be compared to a predefined layout of the furniture unit to identify which of the inventory items are missing from the empty location.

At 812, a database accessed by the system may be updated to indicate the status of the inventory items (e.g., absence and/or presence of the inventory items). For example, updating the database can include updating a record associated with a status of the inventory item to indicate whether the inventory item is present or absent from the furniture unit.

At 814, the first technique 800 may output information indicative of the updated database record. For example, the system may output information to a dashboard of a software application (e.g., the software application 108), which may, for example, be included in a GUI of the software application.

At 816, the first technique 800 may further generate an alert based on the presence and/or absence of inventory items. This alert may be transmitted to a user device as a push notification, text message, or interface banner. In some cases, the alert may also include the item name and location of the furniture unit. Additionally, the system may transmit the alert to an inventory management system for storage, display, or order initiation. For example, the system may automatically generate a restock request for the furniture unit when an alert is triggered. In another example, the system may automatically place a purchase order for the inventory item absent from the furniture unit.

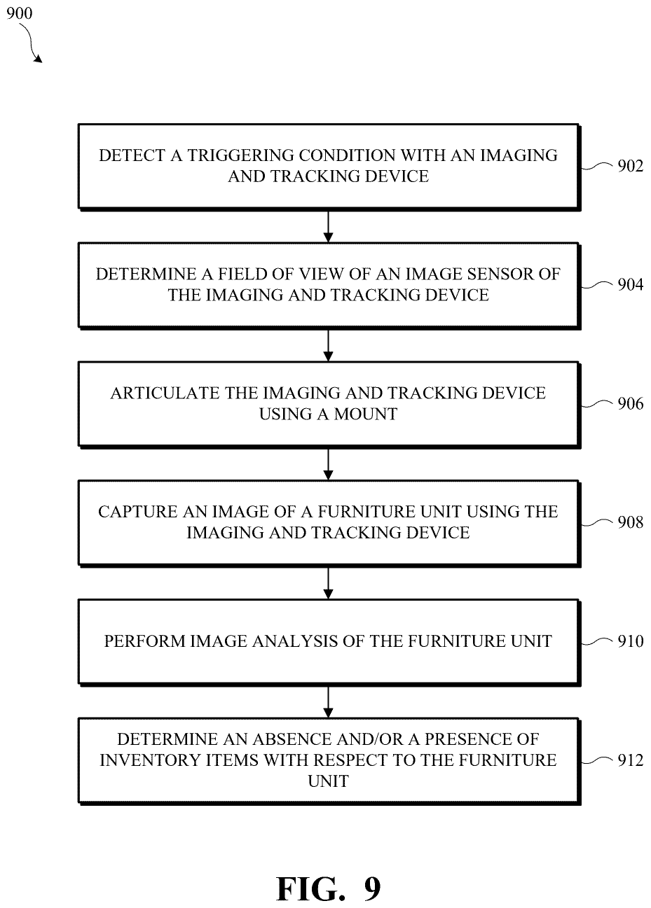

FIG. 9 is a flowchart showing an example of a second technique 900 for detecting the presence and/or absence of inventory items using an inventory imaging and tracking intelligence system. The second technique 900 can be executed using computing devices, such as the systems, hardware, and software described with respect to FIGS. 1-8. The second technique 900 can be performed, for example, by executing a machine-readable program or other computer-executable instructions, such as routines, instructions, programs, or other code. The steps, or operations, of the second technique 900 or another technique, method, process, or algorithm described in connection with the implementations disclosed herein can be implemented directly in hardware, firmware, software executed by hardware, circuitry, or a combination thereof.

At 902, a triggering condition may be detected by an imaging and tracking device and/or a mount (e.g., the mount 406 of FIGS. 4A and 4B) that may couple the imaging and tracking device to a furniture unit, whereby the imaging and tracking device may be positioned to capture one or more images of the furniture unit and the inventory items therein. The imaging and tracking device may be any one of the imaging and tracking devices described herein with respect to FIGS. 1-8. The furniture unit can be any one of the furniture units described herein or another furniture unit. As described herein, the mount may facilitate articulation of the imaging and tracking device with respect to the furniture unit to adjust a field of view of the image sensor prior to and/or after capturing an image, as discussed herein. The triggering condition may be detected using the techniques described above, such as by using one or more sensors (e.g., a motion sensor, an accelerometer, another sensor, or the like).

At 904, a field of view of an image sensor of the imaging and tracking device may be determined. As described herein, the imaging and tracking device may include an image sensor (e.g., the image sensor 306 shown in FIG. 3), which may have a field of view (e.g., the field of view 312 of the image sensor 306 shown in FIG. 3) to capture images of the furniture unit and the inventory items therein. As such, the field of view of the image sensor may be positioned such that the furniture unit or a portion thereof (e.g., when multiple imaging devices may be used) is located within the field of view.

Determining the field of view of the image sensor may include determining whether the field of view is obstructed or otherwise blocked such that the images captured by the imaging and tracking device may not clearly reflect the furniture unit and/or the inventory items therein. For example, a bulky or oversized inventory item may be positioned within the furniture unit close to the imaging and tracking device such that the bulky or oversized inventory item may obstruct the field of view and block the imaging and tracking device from capturing the inventory items located behind the bulky or oversized inventory. Similarly, the imaging and tracking device may be inadvertently hit when storing and/or retrieving inventory items from the furniture unit, thereby moving the position of the imaging and tracking device and thus changing the field of view (e.g., changing the field of view such that the furniture unit is no longer properly located within the field of view). As a result, when the image is captured by the imaging and tracking device, the system may be unable to accurately identify the inventory items contained within the furniture unit.

As such, the field of view of the image sensor may be determined to identify whether obstruction or blocking within the image is present. For example, the system may be configured to analyze an initial image captured by the imaging and tracking device to identify whether obstruction or blocking is present. That is, the imaging and tracking device may capture an initial image (e.g., based upon a triggering condition detected at 902) and the system may analyze the initial image to detect one or more predefined conditions which may correspond to an obstruction or blocking. In an implementation, the system may implement an image analysis similar to the image analysis utilized to determine the presence and/or absence of inventory items, such as by using pixel quantification or other computer vision-based analyses.

For example, the system may be configured to compare the size of a particular inventory item (e.g., the size of a bounding box associated with an inventory item based upon pixel quantification) against a predefined threshold (e.g., a predefined size threshold) for that particular inventory item. If the size of the particular inventory item exceeds the predefined threshold (e.g., is larger than the predefined size threshold), it may be determined that the particular inventory item is positioned too closely in the foreground of the image captured and thereby obstructing the view of the furniture unit. Alternatively, or additionally, the system may be configured to determine whether the size of a particular inventory item occupies an abnormally large proportion of the image (i.e., an abnormally large portion of the furniture unit). For example, a size of the particular inventory item may be compared to a predefined threshold, which may correspond to a proportion threshold (e.g., a percentage). If the particular inventory item exceeds the proportion threshold (e.g., the particular inventory items account for greater than 60% of the image frame, greater than 70% of the image frame, etc.), the system may determine that the inventory item is obstructing the view of the furniture unit.

Other techniques may also be implemented to determine the field of view and identify obstructions or blockages therein. For example, occlusion analysis may be implemented by analyzing regions of the captured image where a closer inventory item occludes areas beyond it based upon depth discontinuities. Additionally, or alternatively, the system may be configured to monitor edge and/or texture density of the inventory items to identify whether inventory items have a low edge density within a large region of the captured image, which may indicate a possible obstruction. Moreover, the system may be configured to perform blind spot detection, which may be used to determine whether a portion of the furniture unit that is expected to be visible (e.g., a leg, support, hinge, etc.) is in fact visible. If the portion of the furniture unit that is expected to be visible is missing or covered, an obstruction may exist.

If the field of view of the image sensor is determined to not be obstructed or otherwise blocked, the system may capture an image at 908 and/or utilize an initial image captured at 904 that is used to determine the field of view of the image sensor. However, if the field of view of the image sensor is determined to be obstructed or otherwise blocked, the imaging and tracking device may be articulated at 906 using the mount. For example, one or more articulating devices of the mount, such as those described herein, may may translate (e.g., slide) and/or rotate (e.g., pan and/or tilt) the imaging and tracking device to move the image sensor and thus adjust the field of view. In some implementations, a user may manually operate the mount via a user interface (e.g., the client 116 of FIG. 1) to adjust the field of view. In some implementations, the one or more articulating devices may automatically move, such as based upon a command communicated to the one or more articulating devices from a server (e.g., the server 106 operating the software application 108, as shown in FIG. 1). For example, the server may operate a software application to determine whether the field of view is blocked. If the field of view is blocked, the server may automatically communicate to mount an operating condition, which may trigger the one or more articulating devices to move the imaging and tracking device to one or more predefined locations (e.g., alternative locations). The field of view may then be determined once again at 904 (e.g., to determine whether the obstruction or blockage still exists) until the obstruction or blockage is no longer present.

At 908, the imaging and tracking device may capture an image of the furniture unit (e.g., a second image of the furniture unit if an initial image is captured at 904 to determine the field of view of the image sensor). The image may be captured in response to the triggering condition. The image may be a still image or a frame from a video feed.

At 910, the second technique 900 may perform image analysis. The image analysis may be similar to the operations of the first technique 800 shown in FIG. 8, such as at 802-810. For example, the image analysis may be any type of computer vision-based analysis that may identify, classify (e.g., sort, group, etc.), count, estimate a volume of, or a combination thereof the inventory items, such as by performing pixel quantification of the furniture unit using a trained machine learning model, to determine the presence and/or absence of the inventory items.

At 912, the second technique 900 may determine the absence and/or presence of the inventory items with respect to the furniture unit. Such a determination may be similar to the operations of the first technique 800 shown in FIG. 8, such as at 802-810.

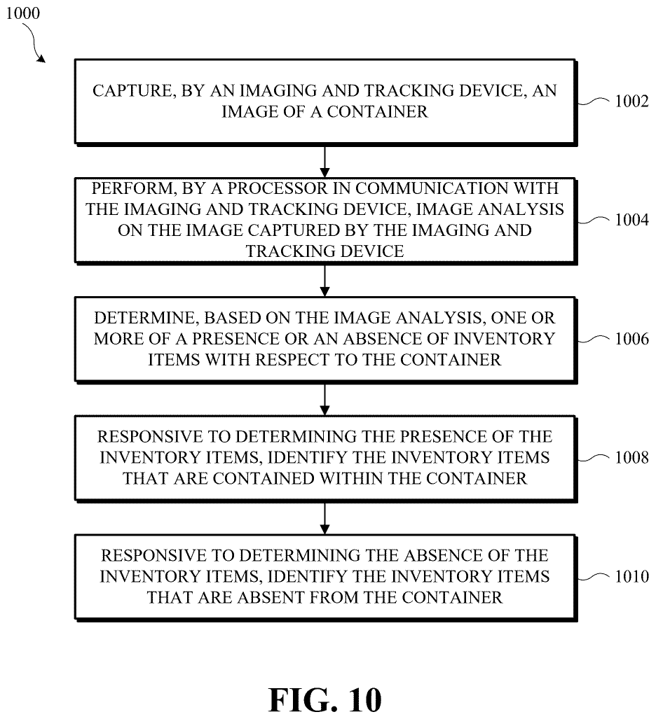

FIG. 10 is a flowchart showing an example of a third technique 1000 for detecting the presence and/or absence of inventory items using an inventory imaging and tracking intelligence system. The third technique 1000 can be executed using computing devices, such as the systems, hardware, and software described with respect to FIGS. 1-9. The third technique 1000 can be performed, for example, by executing a machine-readable program or other computer-executable instructions, such as routines, instructions, programs, or other code. The steps, or operations, of the third technique 1000 or another technique, method, process, or algorithm described in connection with the implementations disclosed herein can be implemented directly in hardware, firmware, software executed by hardware, circuitry, or a combination thereof.

At 1002, the third technique 1000 may capture, by an imaging and tracking device, an image of a container. The imaging and tracking device may be any one of the imaging and tracking devices described herein with respect to FIGS. 1-9. While a container is described herein, the third technique 1000 may also be implemented in any type of furniture unit.

At 1004, a processor in communication with the imaging and tracking device may perform image analysis on the image captured by the imaging and tracking device. The processor may be any processor described herein, such as a processor of the server 106 shown in FIG. 1, the processor 206 of FIG. 2, or the processor 308 of FIG. 3. The image analysis may be similar to the image analysis performed at 910 of the second technique 900 shown in FIG. 9. For example, the image analysis may be any type of computer vision-based analysis that may identify, classify (e.g., sort, group, etc.), count, estimate a volume of, or a combination thereof the inventory items, such as by performing pixel quantification of the furniture unit using a trained machine learning model.

At 1006, the third technique 1000 may determine, based on the image analysis, one or more of a presence or an absence of inventory items with respect to the container. That is, using the techniques described herein, the third technique 1000 may determine whether one or more inventory items are present within the container and/or are absent from the container.

At 1008, responsive to determining the presence of the inventory items within the container, the third technique 1000 may identify the inventory items that are contained within the container using any of the techniques described herein.

At 1010, responsive to determining the absence of the inventory items, the third technique 1000 may identify the items that are absent from the container using any of the techniques described herein.

The implementations of this disclosure can be described in terms of functional block components and various processing operations. Such functional block components can be realized by a number of hardware or software components that perform the specified functions. For example, the disclosed implementations can employ various integrated circuit components (e.g., memory elements, processing elements, logic elements, look-up tables, and the like), which can carry out a variety of functions under the control of one or more microprocessors or other control devices. Similarly, where the elements of the disclosed implementations are implemented using software programming or software elements, the systems and techniques can be implemented with a programming or scripting language, such as C, C++, Java, JavaScript, assembler, or the like, with the various algorithms being implemented with a combination of data structures, objects, processes, routines, or other programming elements.

Functional aspects can be implemented in algorithms that execute on one or more processors. Furthermore, the implementations of the systems and techniques disclosed herein could employ a number of conventional techniques for electronics configuration, signal processing or control, data processing, and the like. The words “mechanism” and “component” are used broadly and are not limited to mechanical or physical implementations, but can include software routines in conjunction with processors, etc.

Likewise, the terms “system” or “mechanism” as used herein and in the figures, but in any event based on their context, may be understood as corresponding to a functional unit implemented using software, hardware (e.g., an integrated circuit, such as an ASIC), or a combination of software and hardware. In certain contexts, such systems or mechanisms may be understood to be a processor-implemented software system or processor-implemented software mechanism that is part of or callable by an executable program, which may itself be wholly or partly composed of such linked systems or mechanisms.

Implementations or portions of implementations of the above disclosure can take the form of a computer program product accessible from, for example, a computer-usable or computer-readable medium. A computer-usable or computer-readable medium can be any device that can, for example, tangibly contain, store, communicate, or transport a program or data structure for use by or in connection with any processor. The medium can be, for example, an electronic, magnetic, optical, electromagnetic, or semiconductor device.

Other suitable mediums are also available. Such computer-usable or computer-readable media can be referred to as non-transitory memory or media, and can include volatile memory or non-volatile memory that can change over time. A memory of an apparatus described herein, unless otherwise specified, does not have to be physically contained by the apparatus, but is one that can be accessed remotely by the apparatus, and does not have to be contiguous with other memory that might be physically contained by the apparatus.

While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method, comprising:
capturing, by an imaging and tracking device, an image of a container;
performing, by a processor in communication with the imaging and tracking device, image analysis on the image captured by the imaging and tracking device;
determining, based on the image analysis, one or more of a presence or an absence of inventory items with respect to the container;
responsive to determining the presence of the inventory items, identifying the inventory items that are contained within the container; and
responsive to determining the absence of the inventory items, identifying the inventory items that are absent from the container.

2. The method of claim 1, further comprising:
detecting, by the imaging and tracking device, a triggering condition,
wherein the image is captured by the imaging and tracking device in response to the triggering condition.

3. The method of claim 2, wherein the triggering condition includes one or more of motion detection, vibration detection, or weight changes.

4. The method of claim 1, wherein responsive to determining the presence of inventory items, the method further comprises:
determining, based on the image analysis, a distance between the inventory items that are contained within the container.

5. The method of claim 1, wherein determining one or more of the presence or the absence of the inventory items includes:
comparing the image captured by the imaging and tracking device to a predefined configuration of the container, wherein the predefined configuration includes one or more of a predefined quantity of the inventory items or a predefined location of the inventory items.

6. The method of claim 1, wherein identifying the inventory items that are contained within the container includes identifying the inventory items based on one or more features of the inventory items, and wherein the one or more features include at least one of color, shape, size, relative position, a barcode, or a label.

7. The method of claim 6, further comprising:
detecting labels of the inventory items that are contained within the container; and
classifying the inventory items that are contained within the container based on the labels.

8. The method of claim 1, further comprising:
generating an alert; and
transmitting the alert to an inventory management system for one or more of storage, display, or order initiation, wherein the alert includes identification of one or more of the inventory items that are contained within the container or the inventory items that are absent from the container.

9. The method of claim 1, further comprising:
determining a field of view of an image sensor of the imaging and tracking device based on the image analysis on the image captured;
comparing the field of view to a predefined threshold condition;
responsive to determining that the field of view corresponds to the predefined threshold condition, articulating the imaging and tracking device via a mount to adjust the field of view; and
capturing, by the imaging and tracking device, an additional image of the container, wherein the image analysis is performed on the additional image.

10. The method of claim 1, wherein the image analysis includes:
performing, by a machine learning model, pixel quantification of the container; and
applying, using the processor, object detection based on one or more of physical features of the inventory items or data present on the inventory items.

11. The method of claim 10, wherein the data includes one or more of labels or barcodes.

12. A system, comprising:
an imaging and tracking device that is configured for removable mounting to a container via a mount, wherein the imaging and tracking device is configured to capture an image of the container; and
a server device in communication with the imaging and tracking device, wherein the server device comprises a processor configured to:
perform an image analysis on the image captured by the imaging and tracking device;
determine, based on the image analysis, one or more of a presence or an absence of inventory items with respect to the container;
responsive to determining the presence of the inventory items, identify the inventory items that are contained within the container; and
responsive to determining the absence of the inventory items, identify the inventory items that are absent from the container.

13. The system of claim 12, wherein the imaging and tracking device is further configured to detect a triggering condition, and wherein the imaging and tracking device is configured to capture the image in response to the triggering condition.

14. The system of claim 12, wherein responsive to determining the presence of inventory items, the processor is further configured to:
determine, based on the image analysis, a distance between the inventory items that are contained within the container.

15. The system of claim 12, wherein determining one or more of the presence or the absence of inventory items includes:
comparing the image captured by the imaging and tracking device to a predefined configuration of the container, wherein the predefined configuration includes one or more of a predefined quantity of the inventory items or a predefined location of the inventory items.

16. The system of claim 12, wherein identifying the inventory items that are contained within the container includes identifying the inventory items based on one or more features of the inventory items, and wherein the one or more features include at least one of color, shape, size, relative position, a barcode, or a label.

17. The system of claim 12, wherein the mount includes an articulating device that is configured to one or more of translate the imaging and tracking device with respect to the container or rotate the imaging and tracking device with respect to the container.

18. The system of claim 17, wherein the processor is further configured to:
determine a field of view of an image sensor of the imaging and tracking device based on the image analysis on the image captured;
compare the field of view to a predefined threshold condition;
responsive to determining that the field of view corresponds to the predefined threshold condition, articulate the imaging and tracking device via the articulating device to adjust the field of view; and
capture, by the imaging and tracking device, an additional image of the container, wherein the image analysis is performed on the additional image.

19. The system of claim 12, wherein the image analysis includes:
performing, by a machine learning model, pixel quantification of the container, and
applying object detection based on one or more of physical features of the inventory items or data present on the inventory items.

20. A method, comprising:
removably coupling an imaging and tracking device to a container via a mount, wherein the mount includes an articulating device that is configured to articulate the imaging and tracking device;
detecting, by one or more of the imaging and tracking device or the mount, a triggering condition;
responsive to detecting the triggering condition, capturing, by the imaging and tracking device, an image of the container;
comparing, by a processor in communication with the imaging and tracking device, the image captured by the imaging and tracking device to a predefined configuration of the container to determine one or more of a presence or an absence of inventory items with respect to the container, wherein the inventory items are surgical tools, and wherein the predefined configuration includes one or more of a predefined quantity of the inventory items or a predefined location of the inventory items;
responsive to determining the presence of the inventory items, identifying the inventory items that are contained within the container; and
responsive to determining the absence of the inventory items, identifying the inventory items that are absent from the container.
